(19) United States
(12) Patent Application Publication
(10) Pub. No.: US 2006/0270027 A1
(43) Pub. Date: Nov. 30, 2006

(54) High throughput incubation devices and systems

(75) Inventors: Christopher M. Shaw, San Diego, CA (US); Robert C. Downs, La Jolla, CA (US)

Correspondence Address: QUINE INTELLECTUAL PROPERTY LAW GROUP, P.C., P O BOX 458, ALAMEDA, CA, 94501, US

(73) Assignee: IRM LLC, Hamilton (BM)

(21) Appl. No.: 11/140,530

(22) Filed: May 27, 2005

(51) Int. Cl. C12M 1/02

(52) U.S. Cl. 435/303.3; 435/286.7

(57) ABSTRACT

The invention provides efficiently automated incubation devices that reduce the number of powered moving parts in the devices and the amount of air transfer between environments that are internal and external to the devices. Related systems are also provided.

### COPYRIGHT NOTIFICATION

Pursuant to 37 C.F.R. § 1.71(e), Applicants note that a portion of this disclosure contains material that is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### FIELD OF THE INVENTION

This invention relates to laboratory incubators employed to store chemical compounds, cells, mixtures or other materials.

### BACKGROUND OF THE INVENTION

Laboratory incubators are used, e.g., to store chemical compounds, cells, mixtures or other materials in a controlled atmospheric environment. A design for an incubator typically maximizes device storage capacity, while minimizing air transfer between the internal and external environments when sample containers are loaded into and unloaded from the device. Incubators in current use either include a single large door or an internal mechanism. Access to large door units requires opening the single large door, which exposes the entire storage area to the uncontrolled external environment. It is difficult to maintain internal stability (e.g., a selected temperature, a desired humidity level, a gas composition, etc.) when opening such large doors, because large volumes of air transfer between the controlled and uncontrolled environments. In contrast, an internal mechanism incubator includes a single small mechanically actuated door or slot through which sample containers are robotically or manually passed from the external environment. A secondary system, e.g., a robot, disposed in the controlled environment of the incubator then moves the container or other lab-ware to its storage location within the device. Although this method reduces air transfer between controlled and uncontrolled environments, the secondary system disposed in the device is required to move objects within the incubator. Furthermore, breaches to the barrier between controlled and uncontrolled environments of these devices also result when repairs to the secondary system are required.

Accordingly, it would be desirable to provide a simplified incubation device that reduces air exchange between internal and external environments. The invention provides this and a variety of additional features that will become apparent upon complete review of the following disclosure.

### SUMMARY OF THE INVENTION

The present invention relates generally the incubation of objects, such as sample containers or the like. In certain embodiments, for example, the invention provides incubation devices that are structured to minimize air, heat, and other exchanges between environments that are internal to the devices and those that are external to the devices. Among the advantages provided by these devices is greater control over the internal conditions of the devices relative to the control of such conditions in more conventional devices, particularly during processes of accessing the device interiors from external environments. In addition to incubation devices, the invention also provides related systems.

In one aspect, the invention provides an incubation device that includes (a) a housing that comprises at least one opening, and (b) at least one door operably connected to the housing and disposed in or proximal to the opening, which door occludes the opening opening when the door is closed. In some embodiments, the incubation device includes at least one restraining mechanism (e.g., springs, etc.) operably connected to the door. The restraining mechanism is configured to close the door in the absence of an opposing force applied on the door. Typically, the housing comprises at least one additional door that allows access to an interior of the housing. In some embodiments, (b) comprises multiple doors arranged in at least one column and/or at least one row. In certain embodiments, the door lacks an individual actuator mechanism that opens the door. The incubation device also includes (c) a rotatable carousel that comprises at least one shelf. In some embodiments, the shelf comprises a retaining feature that is structured to retain an object when the object is placed on the shelf. Typically, the shelf is capable of moving into alignment with at least a portion of the opening. Optionally, the shelf comprises at least one identification label and/or at least one sensor. In addition, the incubation device also includes (d) a rotational mechanism operably connected to the rotatable carousel. The rotational mechanism is configured to oscillate the rotatable carousel as the rotatable carousel is rotated to thereby agitate an object when the object is disposed on the shelf.

In some embodiments, the incubation device includes (e) a door hold-open mechanism that comprises a member having at least one stop positioned proximal to the door. The stop is structured to move into contact with the door when the door is at least partially open to thereby hold the door open. In some of these embodiments, the stop comprises a prong attached to the member. Typically, the incubation device includes an actuating device that is configured to move the door hold-open mechanism.

In certain embodiments, (b) comprises at least one door pair, and the incubation device comprises (e) at least first and second door hold-open mechanisms. The first door hold-open mechanism comprises a first member having at least a first stop positioned proximal to the first door. The first stop (e.g., a prong attached to the first member, etc.) contacts the first door when the first member moves in a upward direction and the first door is at least partially open to thereby hold the first door open. The second door hold-open mechanism comprises a second member having at least a second stop (e.g., a prong attached to the second member, etc.) positioned proximal to the second door. The second stop contacts the second door when the second member moves in a downward direction and the second door is at least partially open to thereby hold the second door open. Typically, opposing sides of the first and second doors are hinged such that movement of the first member of the first door hold-open mechanism in a first direction causes the first stop to hold the first door open when the first door is at least partially open, and movement of the second member of the second door hold-open mechanism in a second direction, that is opposite from the first direction, causes the second stop to hold the second door open when the second door is at least partially open.

In some embodiments, the incubation device includes at least one actuating device that is configured to move at least one of the door hold-open mechanisms. Typically, the incubation device includes a plurality of door pairs arranged in a vertical column, each of which door pairs comprises a first door and a second door and occludes an opening or a portion of an opening in the housing when the both the first door and the second door pair of the door pair are closed. In certain embodiments, the first door hold-open mechanism comprises multiple stops that are each positioned adjacent to a different first door in which movement of the first member of the door hold-open mechanism results in a stop contacting any first door that is at least partially open, thereby holding open any first door that is at least partially open. Similarly, in some embodiments, the second door hold-open mechanism comprises multiple stops that are each positioned adjacent to a different second door in which movement of the second member in a direction opposite to the direction of movement of the first member results in a stop contacting any second door that is at least partially open, thereby holding open any second door that is at least partially open.

Typically, the incubation device includes at least one controller operably connected to the housing. The controller is generally configured to control one or more internal housing conditions (e.g., temperature, humidity, gas composition, and/or the like).

In some embodiments, the incubation device includes at least one access panel operably connected to the housing. The access panel typically includes the door. In certain embodiments, the access panel is operably connected to the housing via at least one hinge. Optionally, the access panel comprises a gasket, a lock, and/or a latch. In some embodiments, the access panel is disposed in an additional door in the housing.

In another aspect, the invention provides an incubation device that includes (a) a housing that comprises at least one opening, and (b) at least one door pair comprising a first door and a second door in which a top portion of the first door and a bottom portion of the second door are pivotably attached to the housing and the opening is occluded when both the first door and the second door are closed. In addition, the incubation device also includes (c) at least first and second door hold-open mechanisms. The first door hold-open mechanism comprises a first member having at least a first stop (e.g., a prong or the like) positioned proximal to the first door. The first stop contacts the first door when the first member moves in a upward direction and the first door is at least partially open to thereby hold the first door open. Similarly, the second door hold-open mechanism comprises a second member having at least a second stop (e.g., a prong, etc.) positioned proximal to the second door. The second stop contacts the second door when the second member moves in a downward direction and the second door is at least partially open to thereby hold the second door open.

In some embodiments, the housing further comprises at least one access door that allows access to an interior of the housing. In other exemplary embodiments, at least one access panel is operably connected to the housing. The access panel typically comprises the door pairs. In some of these embodiments, the access panel is operably connected to the housing via at least one hinge. Optionally, the access panel comprises a gasket, a lock, and/or a latch. To further illustrate, the access panel is optionally disposed in an additional door in the housing, e.g., to facilitate access to the interior of the incubation device for maintenance or the like.

The doors of the incubation devices described herein include various embodiments. For example, the doors are typically arranged in at least one column (e.g., one or more vertical columns, etc.) and/or at least one row (e.g., one or more horizontal rows, etc.). Typically, the first and second doors open independently of one another. In some embodiments, the doors comprise a non-linear interface with one another, e.g., such that it is more likely that both doors of the pair are contacted when the interior of the housing is accessed through the doors. In addition, the doors can be fabricated to accommodate a wide range of objects. To illustrate, the doors are structured to accommodate at least one deep-well plate or other sample container in certain embodiments. In some embodiments, the incubation devices of the invention include one or more restraining mechanisms (e.g., springs or the like) operably connected to one or more of the doors. The restraining mechanisms are typically configured to close the doors in the absence of opposing forces applied to the doors. Typically, the doors lack individual actuator mechanisms that open the doors. Moreover, in certain embodiments, the first door and the second door are each rotatably attached to the housing with a hinge. Additionally, movement of the second member of the second door hold-open mechanism in a second direction, that is opposite from the first direction, causes the second stop to hold the second door open when the second door is at least partially open.

In certain embodiments, the incubation devices described herein include a plurality of door pairs arranged in a vertical column, each of which door pairs comprises a first door and a second door and occludes an opening or a portion of an opening in the housing when the both the first door and the second door pair of the door pair are closed. In some embodiments, the plurality of door pairs is arranged in a plurality of columns and/or at least one row. The first door hold-open mechanism typically comprises multiple stops that are each positioned adjacent to a different first door in which upward movement of the first member of the door hold-open mechanism results in a stop contacting any first door that is at least partially open, thereby holding open any first door that is at least partially open. Similarly, the second door hold-open mechanism generally comprises multiple stops that are each positioned adjacent to a different second door in which downward movement of the second member results in a stop contacting any second door that is at least partially open, thereby holding open any second door that is at least partially open.

In some embodiments, the incubation devices include at least one actuating device that is configured to move at least one of the door hold-open mechanisms. Optionally, the first and/or second stop comprises a prong attached to the first and/or second member, respectively.

The incubation devices of the invention include at least one shelf disposed within the housing in certain embodiments. In some embodiments, the shelf is capable of moving into alignment with at least a portion of the opening. In certain embodiments, the shelf is configured to agitate an object when the object is placed on the shelf. Optionally, the shelf comprises at least one identification label, at least one sensor, and/or at least one angled surface that is/are structured to align an object on the shelf, when the object is placed on the shelf. In certain embodiments, incubation devices described herein include multiple shelves arranged in a rotatable carrousel. In some of these embodiments, at least one rotational mechanism is operably connected to the rotatable carrousel. The rotational mechanism is typically configured to rotate the rotatable carrousel in one or more selectable modes. For example, at least one of the selectable modes generally comprises an oscillation of the rotatable carrousel as the rotatable carrousel is rotated to thereby agitate an object when the object is disposed on the shelf. In some embodiments, the shelf comprises a retaining feature that is structured to retain an object when the object is placed on the shelf.

The incubation devices of the invention optionally include at least one controller operably connected to the housing. The controller is typically configured to control one or more internal housing conditions (e.g., temperature, humidity, gas composition, etc.).

In another aspect, the invention provides systems that include the incubation devices described herein. In certain embodiments, a system includes at least one handling apparatus disposed substantially external to the housing. The handling apparatus is generally configured to move objects into and out of the incubation device through the door pairs. Typically, the handling apparatus comprises at least one robotic armature and/or at least one gripper mechanism that is/are structured to grip at least one object. Optionally, the system comprises at least one logic device operably connected at least to the handling apparatus. The logic device typically comprises one or more logic instructions that direct movement of the handling apparatus. In some embodiments, a system includes at least one computer system operably connected to the incubation device. The computer system typically comprises one or more of, e.g., a data input source, a data storage location, a data output device, or the like. The data input source optionally comprises one or more of, e.g., a label reader, an operator input device, an internal housing condition transmitting device, etc. To further illustrate, the data storage location comprises a shelf database in some embodiments, while the data output device optionally comprises one or more of, e.g. a computer monitor, a digital readout, or the like.

In another aspect, the invention provides a method of accessing an incubation device. The method includes (a) contacting a door pairs of the incubation device such that door hold-open mechanisms of the incubation device hold the doors open. In addition, the method also includes (b) placing an object on, or removing the object from, a shelf disposed within the incubation device, thereby accessing the incubation device. Typically, the method includes performing (a) and (b) using a robotic armature of a handling apparatus.

In another aspect, the invention provides a method of agitating an object. The method includes (a) providing an incubation device that houses a rotatable carousel that comprises at least one shelf having the object positioned thereon, and (b) rotating the rotatable carousel. The method also includes (c) oscillating the rotatable carousel as the rotatable carousel is rotated to thereby agitating the object.

### BRIEF DESCRIPTION OF THE DRAWINGS

The nature, goals, and advantages of the invention will become more apparent to those skilled in the art after considering the following detailed description when read in connection with the accompanying drawings in which like reference numbers identify like components throughout the drawings. It will be understood that some or all of the figures may be schematic representations for purposes of illustration and do not necessarily depict the actual relative sizes or locations of the elements shown.

FIG. 1A schematically depicts a front cutaway view of one embodiment of the incubation device of the invention.

FIG. 1B schematically depicts a side cutaway view of one embodiment of the incubation device of the invention.

FIG. 1C schematically depicts a front view of a row of doors according to one embodiment of the incubation device of the invention.

FIG. 1D schematically depicts a front view of a row of pairs of doors according to one embodiment of the incubation device of the invention.

FIG. 2A schematically depicts a top cutaway view of one embodiment of the incubation device of the invention.

FIG. 2B schematically depicts a bottom cutaway view of one embodiment of the incubation device of the invention.

FIG. 2C schematically depicts a top view of a carrousel of an incubation device according to one embodiment of the invention.

FIG. 3 schematically depicts an isolated top cutaway view of vertical columns of shelves disposed proximal to a vertical column of doors according to one embodiment of the invention.

FIG. 4A schematically depicts a front view of one embodiment of the incubation device of the invention.

FIG. 4B schematically depicts a top view of one embodiment of the incubation device of the invention.

FIG. 5A schematically depicts a top cutaway view of one embodiment of the incubation device of the invention.

FIG. 5B schematically depicts a bottom cutaway view of one embodiment of the incubation device of the invention.

FIG. 6A schematically depicts a top cutaway view of a vertical column of doors according to one embodiment of the invention.

FIG. 6B schematically depicts a front cutaway view of a vertical column of doors according to one embodiment of the invention.

FIG. 6C schematically depicts a side cutaway view of a vertical column of doors according to one embodiment of the invention.

FIG. 6D schematically depicts a front view of a vertical column that includes pairs of doors according to one embodiment of the invention.

FIG. 6E schematically depicts a side view of the vertical column of doors shown in FIG. 6D.

FIG. 6F schematically depicts a back view of the vertical column of doors shown in FIG. 6D.

FIG. 6G schematically depicts a front perspective view of the vertical column of doors shown in FIG. 6D.

FIG. 6H schematically depicts a back perspective view of the vertical column of doors shown in FIG. 6D.

FIG. 6I schematically depicts a cross-section of the view of the vertical column of doors shown in FIG. 6D.

FIG. 6J schematically depicts a detail from the view of the vertical column of doors shown in FIG. 6E.

FIG. 6K schematically depicts another detail from the view of the vertical column of doors shown in FIG. 6E.

FIG. 6L schematically depicts a cross-section of the view of the vertical column of doors shown in FIG. 6D.

FIG. 6M schematically depicts a detail from the view of the vertical column of doors shown in FIG. 6F.

FIG. 6N schematically depicts a perspective view of the vertical column of doors shown in FIG. 6D along with door-hold open mechanisms.

FIG. 6O schematically depicts a front view of the vertical column of doors shown in FIG. 6D along with door-hold open mechanisms.

FIG. 6P schematically depicts a side view of the vertical column of doors shown in FIG. 6D along a door-hold open mechanism.

FIG. 6Q schematically depicts a front perspective view of the vertical column of doors shown in FIG. 6D along with door-hold open mechanisms.

FIG. 6R schematically depicts a back perspective view of the vertical column of doors shown in FIG. 6D along with door-hold open mechanisms.

FIG. 6S schematically depicts a detail from the view of the vertical column of doors shown in FIG. 6O.

FIG. 6T schematically depicts a cross-section of the view of the vertical column of doors shown in FIG. 6O.

FIG. 6U schematically depicts a detailed partial cutaway from the view of the vertical column of doors shown in FIG. 6O.

FIG. 7 schematically depicts a gripper of a robotic armature interfacing with a door in a vertical column of doors of an incubation device in one embodiment of the invention.

FIG. 8A schematically depicts a side cutaway view of a vertical column of shelves according to one embodiment of the invention.

FIG. 8B schematically depicts a front cutaway view of a vertical column of shelves according to one embodiment of the invention.

FIG. 8C schematically depicts a top cutaway view of one embodiment of a shelf of the invention.

FIG. 8D schematically depicts a bottom cutaway view of one embodiment of a shelf of the invention.

FIG. 8E schematically depicts a top cutaway view of one embodiment of a shelf of the invention with a sample container.

FIG. 8F schematically depicts a front cutaway view of one embodiment of a shelf of the invention.

FIG. 8G schematically depicts a top cutaway view of a shelf according to one embodiment of the invention.

FIG. 8H schematically depicts a perspective view of a shelf having a retaining feature according to one embodiment of the invention.

FIG. 8I schematically depicts a side elevational view of a shelf having a retaining feature according to one embodiment of the invention.

FIG. 8J schematically depicts a front cutaway view of a shelf having a retaining feature according to one embodiment of the invention.

FIG. 8K schematically depicts a top cutaway view of a shelf having a retaining feature according to one embodiment of the invention.

FIG. 8L schematically depicts a perspective view of a shelf having retaining features according to one embodiment of the invention.

FIG. 9 is a block diagram illustrating one embodiment of a computer system of an incubation device.

FIG. 10 is a block diagram illustrating one embodiment of a computer system of an incubation device.

### DETAILED DESCRIPTION

Incubation Devices and Systems

Before describing the invention in detail, it is to be understood that this invention is not limited to particular devices or systems, which can vary. It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which the invention pertains.

Incubation devices of the invention typically include a housing with a plurality of doors (i.e., at least two doors) disposed in, e.g., an access panel located on a side (e.g., front side, etc.) of the incubator. Typically, a sample handling apparatus located outside the incubator is used to open individual doors located in the access panel as it loads or unloads sample containers into or out of the incubator. This reduces the air exchange between the external environment and the internal environment of the incubation device along with limiting the moving parts within the interior of the incubation device. As a result, the incubation devices of the invention provide a controlled environment for maintaining parameters, such as humidity, temperature, gas conditions (e.g., CO₂, N₂, or other gas levels).

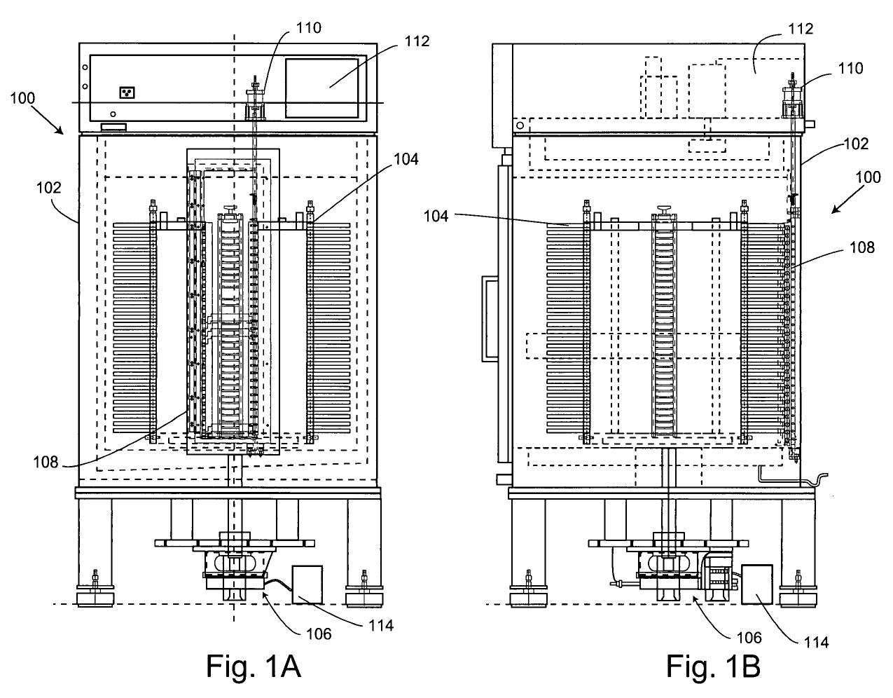

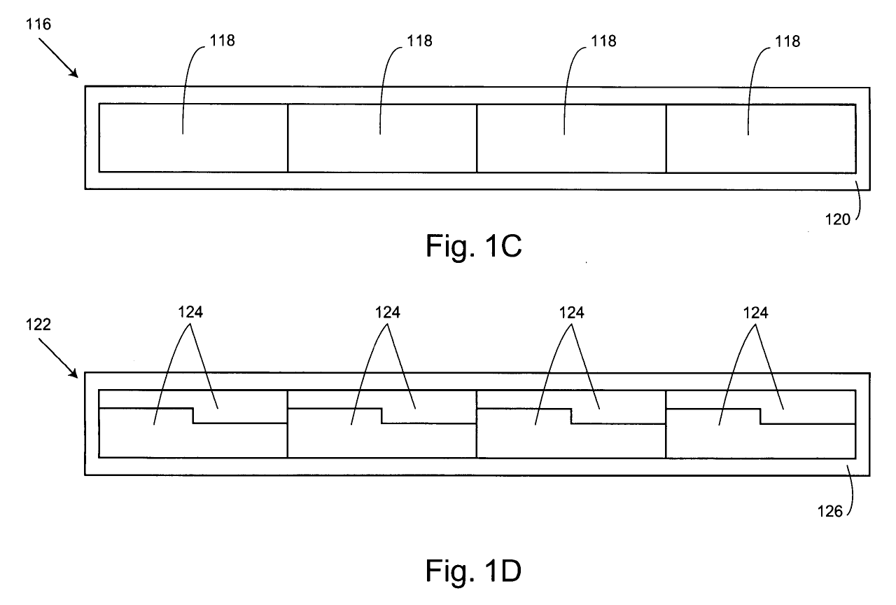

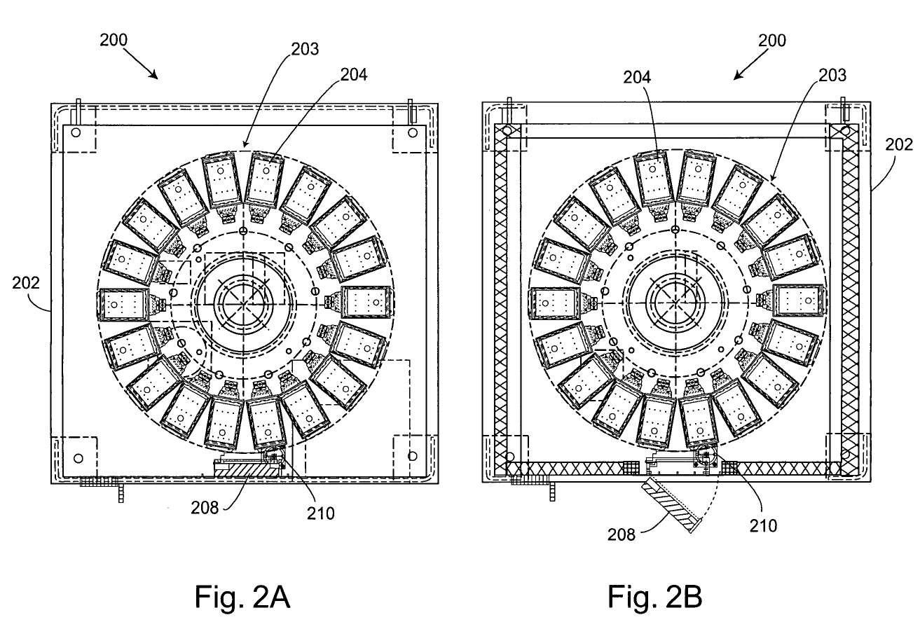

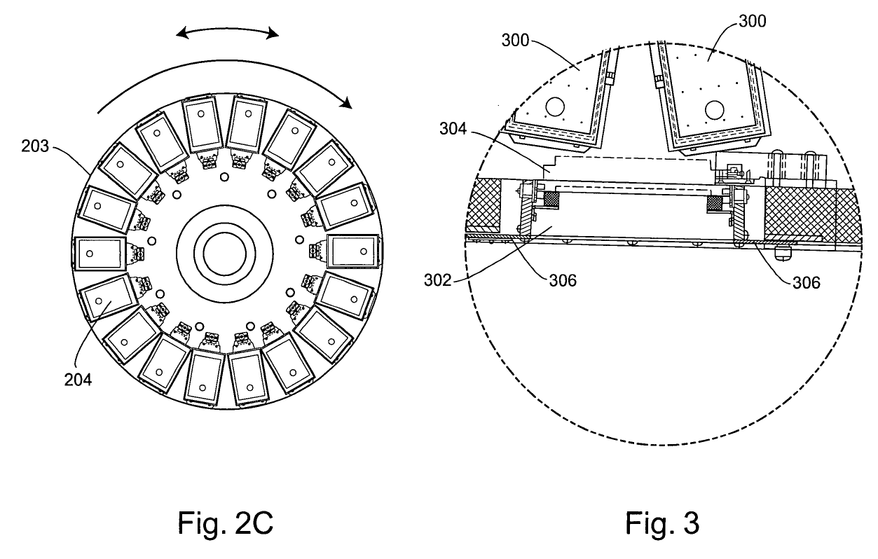

One embodiment of an incubation device of the invention is illustrated schematically in FIG. 1. FIG. 1A schematically depicts a front cutaway view of one embodiment of incubation device 100. As shown, incubation device 100 includes housing 102 having carrousel with vertical columns of shelves 104 disposed in housing 102. As used herein, the term “vertical” refers to a plane that is approximately perpendicular to a plane of a horizontal or supporting surface, such as a shelf or the like. Rotational mechanism 106 (shown as an external motor) is operably connected to carrousel 104 to rotate selected vertical columns of carrousel 104 into alignment with vertical column of doors 108. In certain embodiments, rotational mechanisms are configured to rotate the rotatable carrousels in one or more selectable modes. To illustrate, one exemplary selectable mode includes an oscillation (e.g., a side-to-side motion, etc.) of rotatable carrousels as the rotatable carrousels are rotated, e.g., to agitate containers or other objects disposed on the shelves of the carrousels. An example of this type of oscillation is schematically depicted in FIG. 2C, which shows carrousel 203 from a top view. As shown, the accompanying larger, unidirectional arrow represents the general or overall direction of rotation of carrousel 203, while the accompanying smaller, bi-directional arrow represents the concurrent side-to-side motion of carrousel 203, which effects agitation of objects positioned on shelves 204 of carrousel 203. The general direction of rotation of carrousel 203 can also be opposite from that shown in FIG. 2C. Other approaches to agitating objects placed on incubation device shelves are also optionally utilized. In some embodiments, for example, shelves are configured to tilt or rock from side-to-side, from front-to-back, and/or the like to effect the agitation of objects placed on the shelves. Typically, controller 114 controls rotation of carrousel 104 via rotational mechanism 106, e.g., in these selectable modes. Incubation device 100 also includes controller 112, which controls one or more internal housing conditions. FIG. 1A also schematically illustrates door hold-open mechanism 110 that includes a member (e.g., a rod, a column, a pole, a slat, a bar and the like) having a plurality of prongs (or a series of pins or other stops) for holding accessed doors of vertical column of doors 108 open, as described herein. Although not shown, a sample container handling apparatus (e.g., a robot having a robotic armature, etc.) for inserting and removing sample containers into and out of housing 102 is optionally included as part of a system that includes incubation device 100. Systems are described further below. FIG. 1B schematically depicts incubation device 100 from a side cutaway view.

The doors of the devices described herein are optionally arranged in various configurations. In some embodiments, for example, doors are arranged in columns (e.g., vertical columns, etc.) or rows (e.g., horizontal rows, etc.). As used herein, the term “horizontal” refers to a plane that is approximately parallel to a plane of a supporting surface. To illustrate, FIG. 1C schematically depicts a front view of horizontal row of doors 116 according to one embodiment of the invention. As shown, horizontal row of doors 116 includes individual doors 118 disposed in access panel 120. In another exemplary embodiment, FIG. 1D schematically illustrates a front view of horizontal row of pairs of doors 122. As shown, horizontal row of pairs of doors 122 includes pairs of doors 124 disposed in access panel 126. Incubation device embodiments that include pairs of doors are described further below.

As referred to above, a rotating vertical carrousel with multiple columns (commonly referred to as “hotels”) and multiple shelves is typically located inside the incubation devices described herein. To further illustrate, FIG. 2A schematically depicts a top cutaway view of incubation device 200, while FIG. 2B schematically depicts a bottom cutaway view of incubation device 200 according to one embodiment of the invention. Incubation device 200 includes carrousel 203 with a plurality of shelves 204 disposed in housing 202. A rotational mechanism (not shown) is operably connected to carrousel 203 to rotate selected vertical columns of carrousel 203 (e.g., about an Z-axis) into alignment with vertical column of doors 208. As used herein, the “Z-axis” refers to an axis in a three-dimensional rectangular coordinate system that is substantially perpendicular to a horizontal plane and approximately perpendicular to both the X- and Y-axes. Incubation device 200 also includes door hold-open mechanism 210 that includes a member (e.g., a rod, a column, a pole, a slat, a bar and the like) having a plurality of stops (shown as prongs) for holding accessed doors of vertical column of doors 208 open. In one embodiment, vertical column of doors 208 is hinged to housing 202, which provides the ability to open or close vertical column of doors 208. FIG. 2A schematically depicts vertical column of doors 208 in a closed position, while FIG. 2B schematically depicts vertical column of doors 208 in an open position.

FIG. 3 schematically depicts an isolated top cutaway view of vertical columns of shelves 300 disposed proximal to vertical column of doors 302. As shown, door 304 is depicted open to show the orientation relative to vertical columns of shelves 300. Rotation (e.g., about a Z-axis) of the carrousel aligns one of the shelves 300 with door 304, so that a sample handling apparatus (e.g., robotic armature comprising a gripper mechanism) or the like, can place a sample container or other object on the shelf, or remove such an item from the shelf. Typically, vertical column of doors 302 is disposed within a vertical access panel, which optionally includes gasket 306.

As referred to above, the incubation devices of the invention optionally include access panels (e.g., vertical access panels, horizontal access panels, etc.), which are typically located on the sides (e.g., front sides) of the devices. In some embodiments, access panels are attached to device housings via hinges. An open access panel provides access to a plurality of shelves in a carrousel and the interior compartment of the particular incubation device. Optionally, the access panel includes a gasket to further seal the interior environment of the given incubation device from the exterior environment and a lock, latch, and/or other mechanism to maintain the access panel in a closed position when desired.

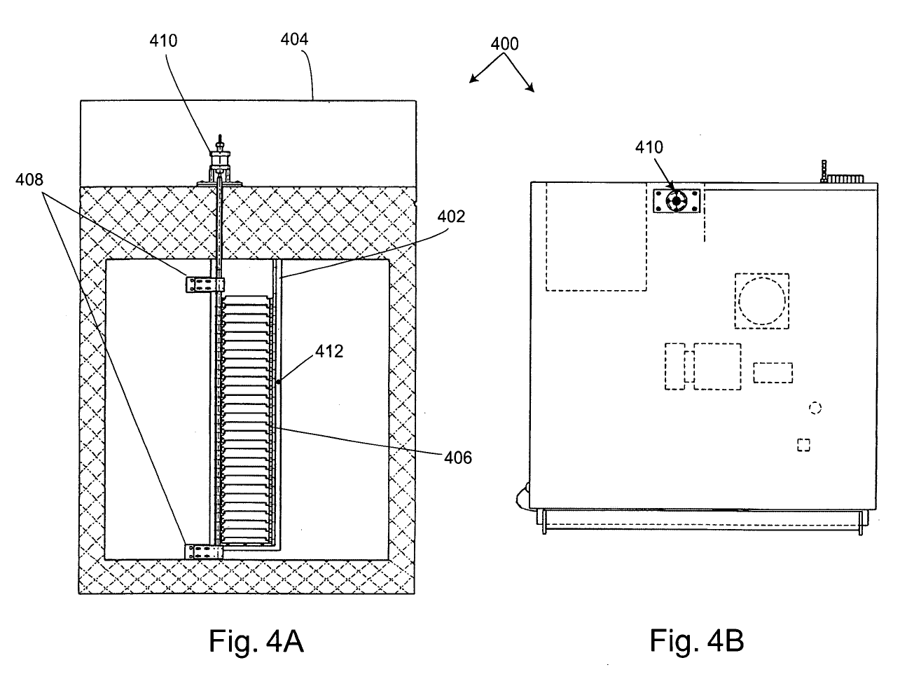

FIG. 4A schematically depicts a front view of incubation device 400 according to one embodiment of the invention. As shown, access panel 402 is disposed in a surface of device housing 404. Access panel 402 includes vertical column of doors 406 and is attached to device housing 404 by hinges 408. Although hinges 408 are depicted on one side of access panel 402, hinges 408 can be placed on any side of access panel 402. Access panel 402 includes lock 412. A portion of door hold-open mechanism 410 is also illustrated. FIG. 4B schematically depicts a top view of incubation device 400.

In another embodiment, access panels are disposed on surfaces of additional doors (e.g., maintenance doors) of device housings. Alternatively, access panels can be disposed on one side of the housings and additional doors can be disposed on the same or different sides of the housings. These additional doors optionally include a gasket to seal the interior environments of the incubation devices from the external environments and a lock, latch, or other mechanism to maintain the doors in closed positions when desired.

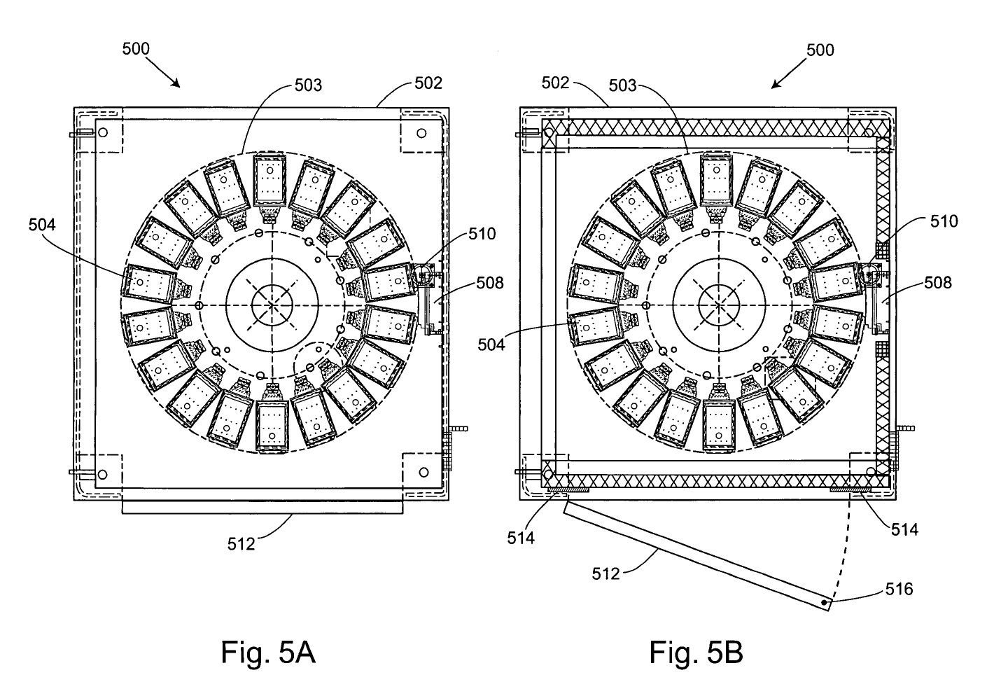

FIGS. 5A and 5B schematically depict incubation device 500 according to one embodiment of the invention. FIG. 5A depicts a top cutaway view and FIG. 5B depicts a bottom cutaway view of incubation device 500. Housing 502 includes vertical column of doors 508, an additional door (depicted as maintenance door 512), carrousel 503 with a plurality of shelves 504 disposed within housing 502. Incubation device 500 also includes door hold-open mechanism 510 that includes a member (e.g., a rod, a column, a pole, a slat, a bar and the like) having a plurality of stops (e.g., prongs, pins, etc.) for holding accessed doors of vertical column of doors 508 open. The interior of housing 502 is accessible via vertical column of doors 508 and/or maintenance door 512. For example, carrousel 503 can be removed or placed in housing 502 via maintenance door 512. Access to the interior of housing 502 can also be used, e.g., to clean the interior of housing 502. FIG. 5B depicts (an open) maintenance door 512 attached to one side of housing 502, via a hinge. As shown, maintenance door 512 also includes gasket 514 and lock 516.

Access panels typically include individual doors that open and provide access to a single sample container or other object disposed in an incubation device described herein. The pitch or spacing of the doors is typically about the same pitch as the storage shelves of a vertical carrousel disposed in the device housings. There is typically one door for every shelf in a given hotel. As a carrousel rotates, different shelves present themselves to the doors of the access panel.

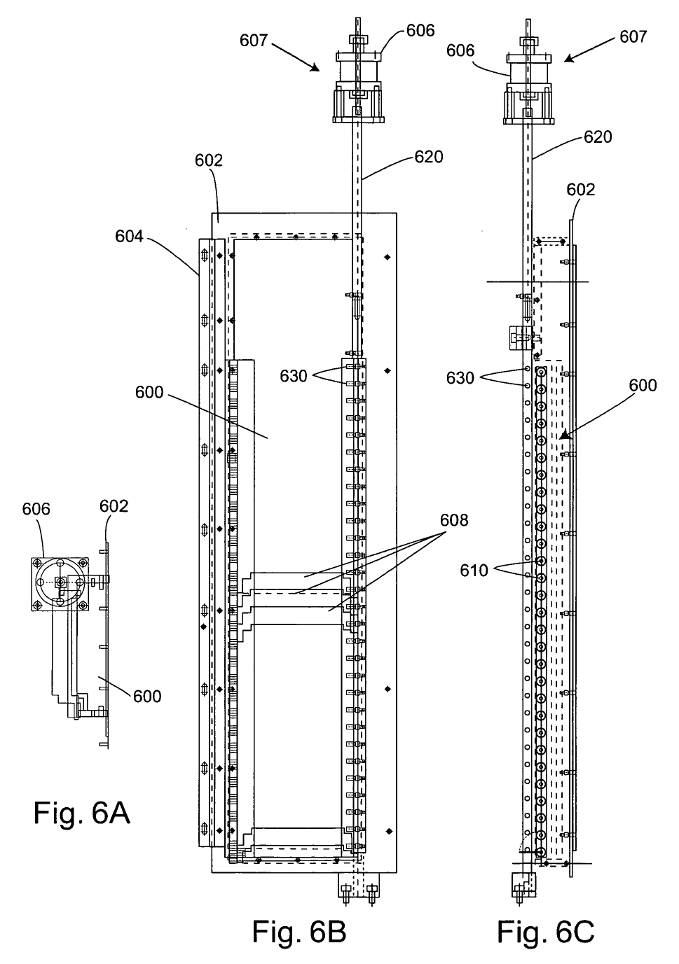

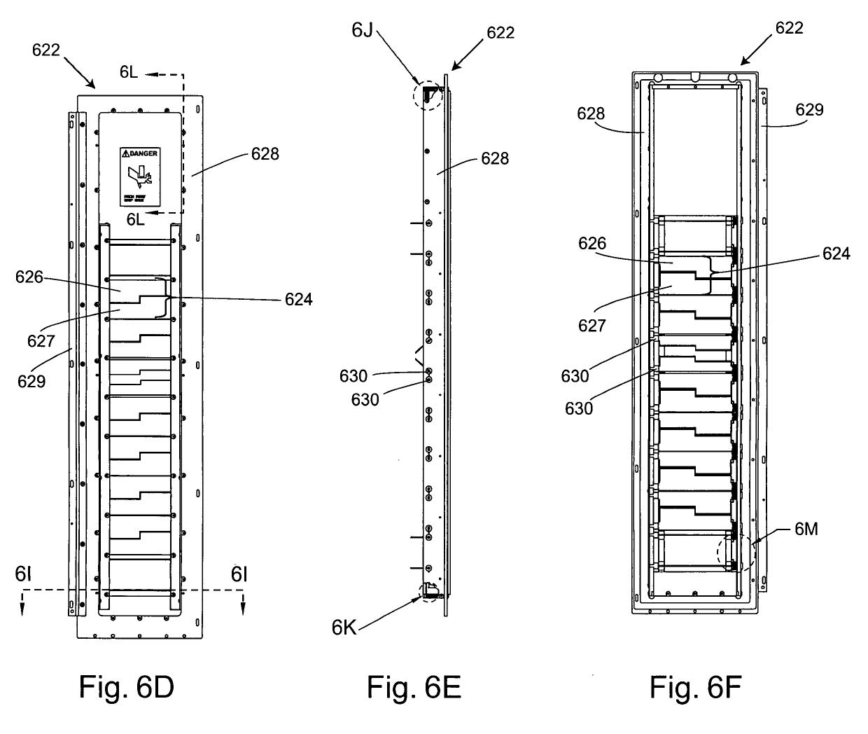

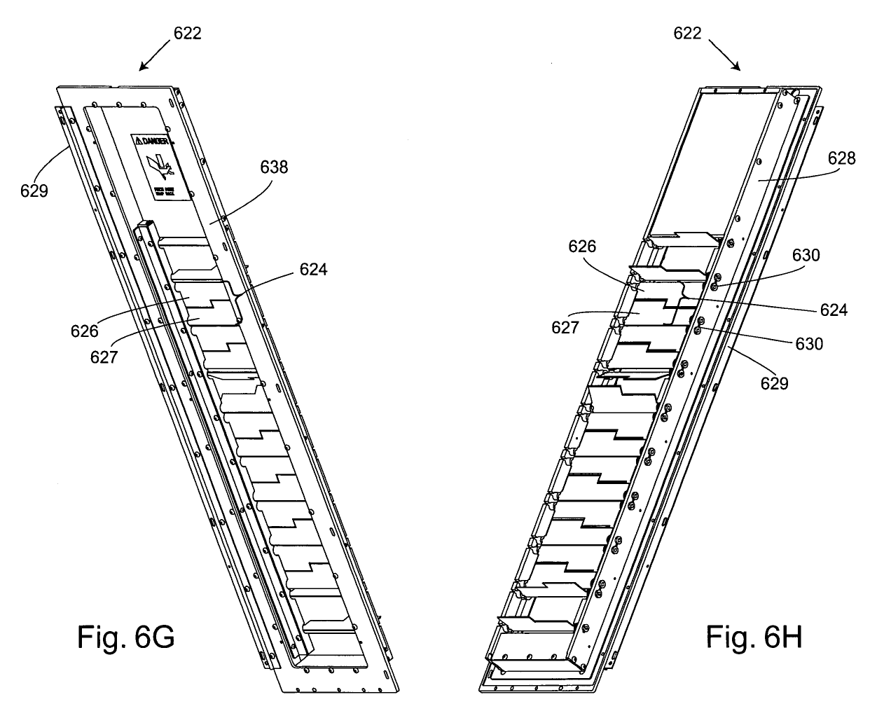

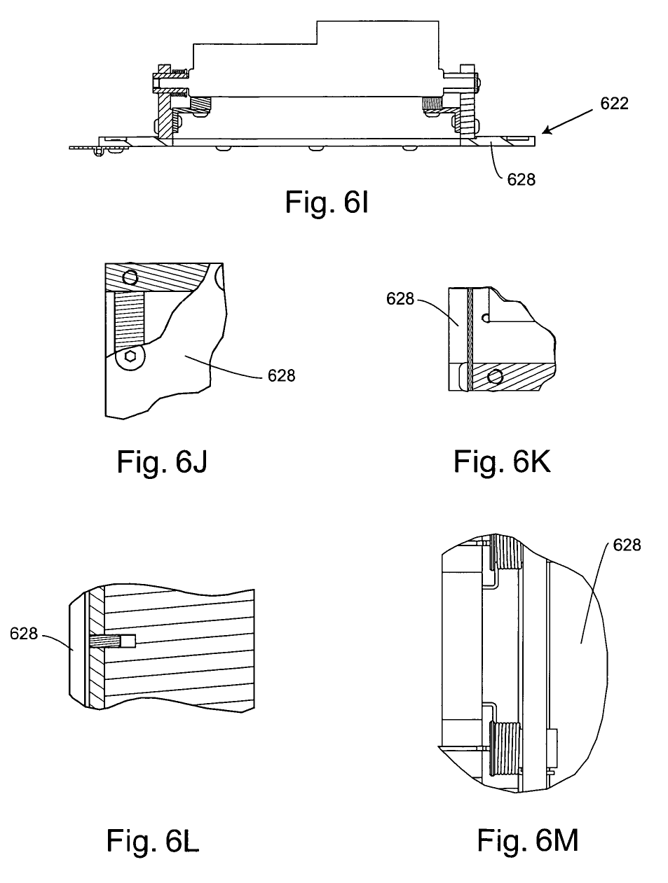

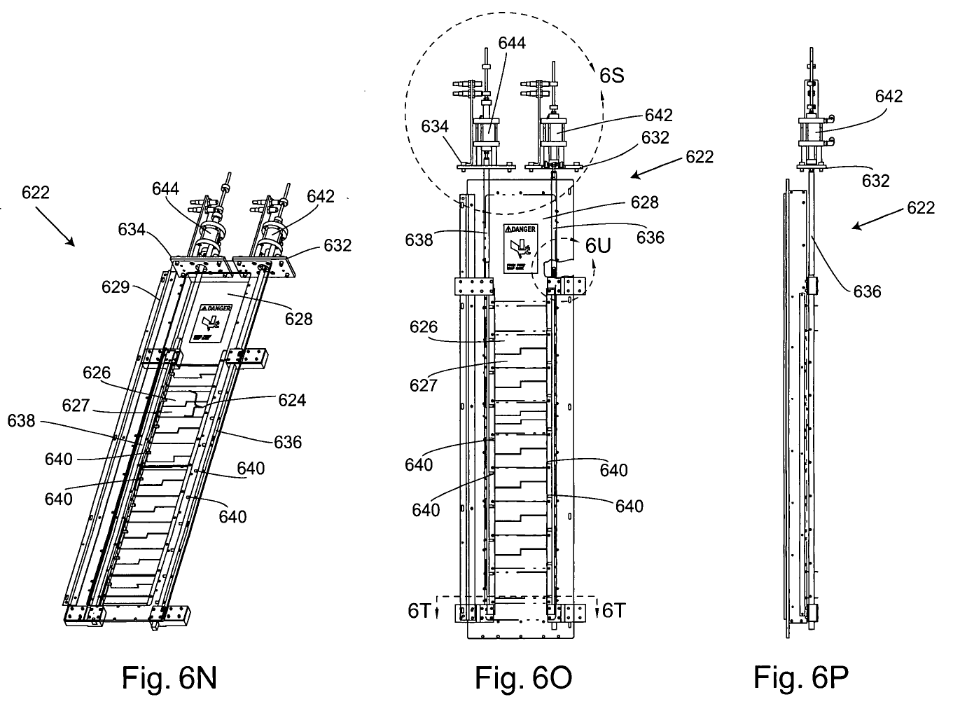

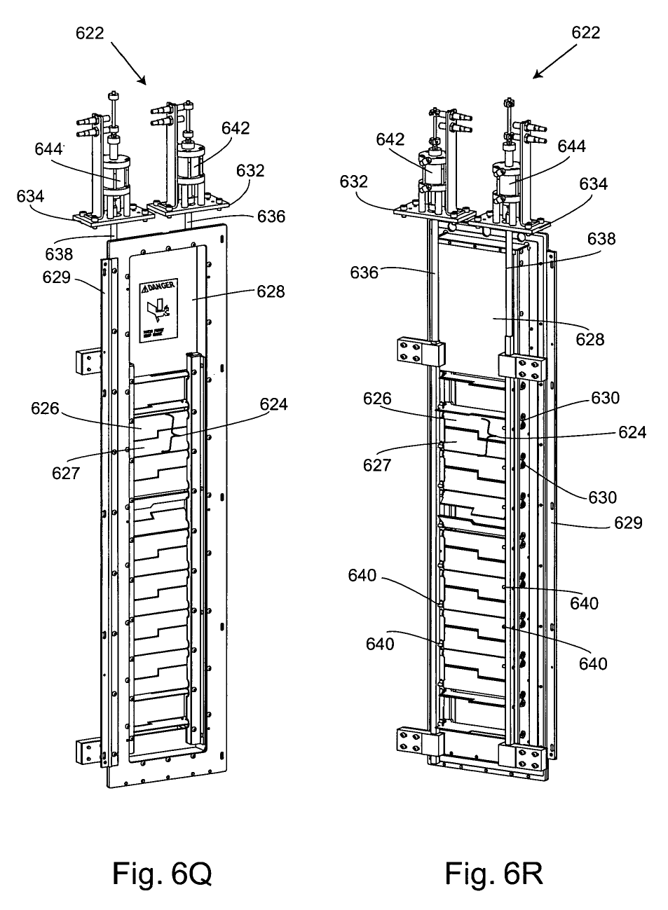

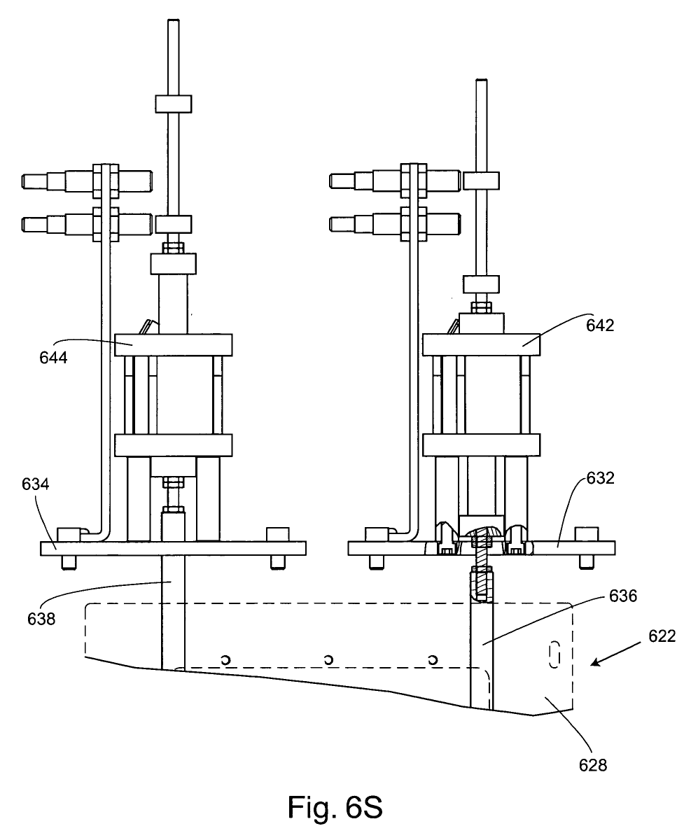

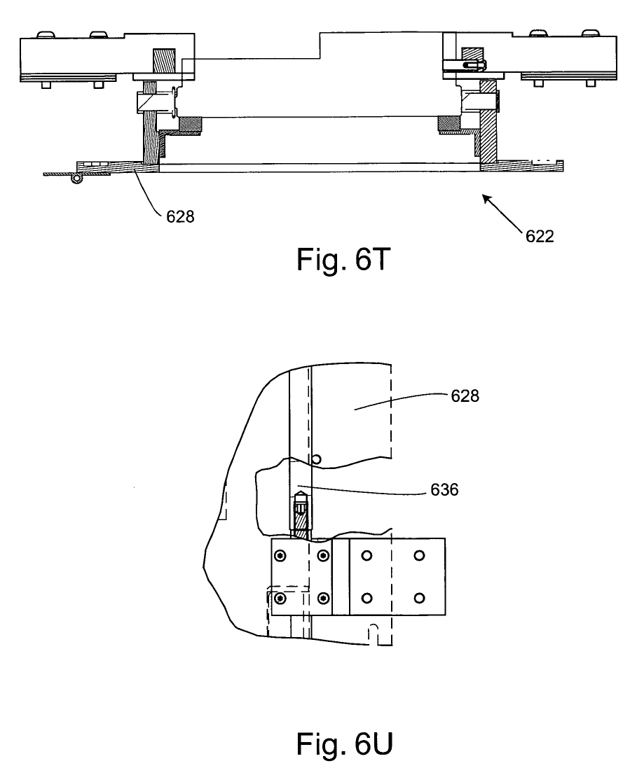

Incubation devices optionally include restraining mechanisms (e.g., spring mechanisms, etc.) to keep doors shut (e.g., similar to a mechanism on a VCR, automotive cassette player, etc.). FIG. 6A schematically depicts a top cutaway view of vertical column of doors 600 disposed in incubation device access panel 602. FIG. 6B schematically depicts a front cutaway view of vertical column of doors 600. Incubation device access panel 602 is typically attached to an incubation device (not shown) by hinge 604, which affords access to the interior of a device housing by opening access panel 602. As also shown, door hold-open mechanism 607 is disposed relative to vertical column of doors 600 to hold doors open, e.g., when aligned shelves are being accessed by, e.g., a sample handling apparatus (e.g., a gripper mechanism of a robotic armature, etc.). Only three doors 608 are illustrated in FIG. 6B. FIG. 6C schematically depicts a side cutaway view of vertical column of doors 600. As shown, restraining mechanisms 610 (shown in FIG. 6C as springs) close individual doors in the absence of an applied opposing force, such as an entering gripper mechanism of a robotic armature.

Door hold-open mechanism 607 includes actuating device 606, which moves member 620 up and down in a direction parallel to a longitudinal axis of the vertical column of doors 600 (FIGS. 6B and 6C). Actuating devices are typically positioned atop the housings of incubation devices (see, e.g., actuating device 410 shown in FIG. 4A), but other locations are also suitable. As also shown, one prong 630 is attached to member 620 for each door in the vertical column of doors. Prongs 630 are positioned such that when a door is partially opened by, for example, a robotic gripper mechanism, and member 620 is moved downward (for doors hinged at the bottom), a prong will contact the partially open door and will further open the door and retain the door in an open position. For doors that are hinged at the top, an upward movement of member 620 will result in a prong contacting any partially opened door, thereby further opening the door and retaining the door in the open position. If two or more doors are opened simultaneously (e.g., by two or more robotic gripper mechanisms accessing an incubation device), the single hold-open mechanism can hold open all of the doors due to the presence of a prong adjacent to each of the partially opened doors. A hold-open mechanism of the devices described herein, thus generally involve only a single moving part in these embodiments, yet functions to hold open any of the doors in the column or row of doors.

Referring now to FIGS. 6D-6U, vertical column of doors 622 that includes multiple pairs of doors 624 is shown according to one embodiment of the invention. As shown, each door pairs 624 includes two doors (626 and 627) that together close a portion of the opening disposed through access panel 628. Hinge 629 attaches access panel 628 to the housing (not shown) of an incubation device. In the embodiment shown, doors (626 and 627) of a given door pairs 624 have a non-linear interface with one another (e.g., a zigzag-type interface). As used herein, “non-linear interface” in the context of doors refers to an interface between doors (e.g., where the doors meet or otherwise contact each other) that is not straight across the entire length of the interface. Examples of non-linear interfaces between doors include those that interdigitated, curved, etc. In certain embodiments, non-linear interfaces are used so that it is more likely that both doors of a pair are contacted when the interior of the housing is accessed through the doors, e.g., by a robotic gripper mechanism or otherwise. In other embodiments, interfaces between doors are linear.

As also shown, access panel 628 also includes door hold-open mechanisms 632 and 634 that comprise members 636 and 638, respectively. Each of members 636 and 638 includes a plurality of stops 640 (shown as prongs) that are positioned proximal to door pairs 624. In particular, stops 640 of member 636 are positioned proximal to doors 627 to move into contact with the doors 627 when doors 627 are at least partially open such that doors 627 are held open. Similarly, stops 640 of member 638 are structured to move into contact with the doors 626 when doors 626 are at least partially open so that doors 626 are held open. Stops other than prongs are also optionally utilized. As also shown, door hold-open mechanisms 632 and 634 also comprise actuating devices 642 and 644, respectively, which are configured to move members 636 and 638, respectively.

The doors of the incubation devices described herein are typically designed to open independently of each other. For example, doors 626 and 627 are attached to access panel 628 by hinges 630 such that movement of member 636 of door hold-open mechanism 632 in downward direction causes stops 640 of member 636 to hold doors 627 open when doors 627 are at least partially open. Additionally, movement of member 638 of door hold-open mechanism 634 in an upward direction causes stops 640 of member 638 to hold doors 626 open when doors 626 are at least partially open.

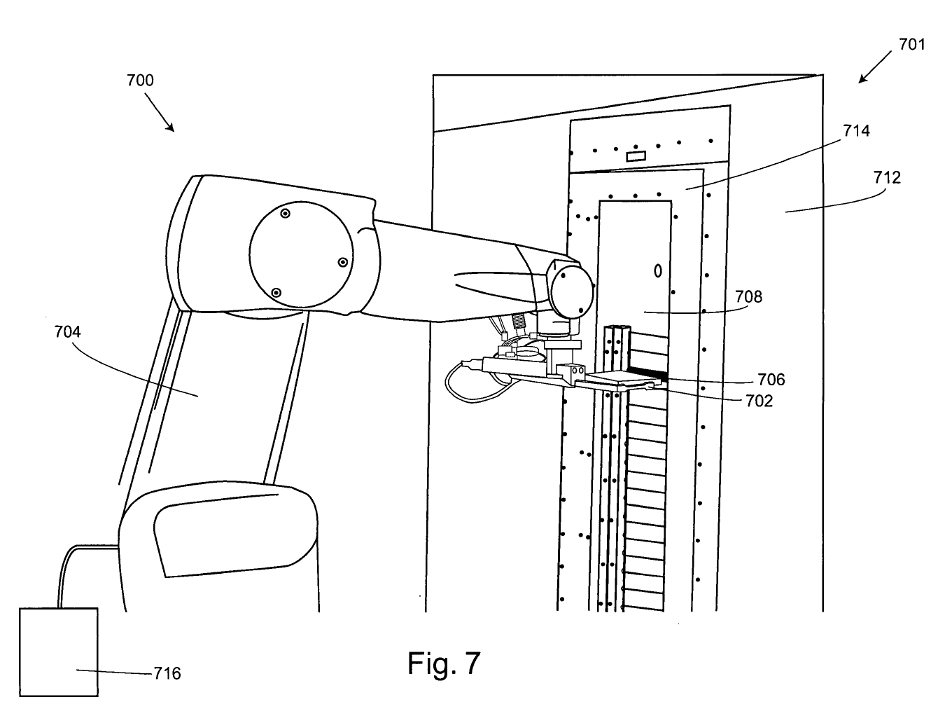

Individual actuators are typically not needed to open doors because a handling apparatus, e.g., robotic armature, typically provides mechanical actuation to open selected doors. Thus, incubation devices need not have any internal mechanism for opening the doors in, e.g., a given vertical column or horizontal row of doors. Since only relatively small doors are open at a time, air exchange between the interior of an incubation device and the outside atmosphere is reduced. FIG. 7 depicts one embodiment of a system of the invention, which includes handling apparatus 700, e.g., a robot, located outside incubation device 701 used to open individual doors 706 on vertical access panel 714. Handling apparatus 700 loads and unloads sample containers or other objects into and out of incubation device 701. FIG. 7 schematically depicts gripper mechanism 702 of robotic armature 704 interfacing with door 706 in vertical column of doors 708 of housing 712 in this exemplary system. Handling apparatus 700 also includes logical device 716 for controlling movement of robotic armature 704. Exemplary robotic gripping devices that are optionally adapted for use in the systems of the invention are described further in, e.g., U.S. Pat. No. 6,592,324, entitled “GRIPPER MECHANISM,” issued Jul. 15, 2003 to Downs et al., and International Publication No. WO 02/068157, entitled “GRIPPING MECHANISMS, APPARATUS, AND METHODS,” filed Feb. 26, 2002 by Downs et al., which are both incorporated by reference.

For example, when a handling apparatus (e.g., a robotic armature) accesses a sample container or other object behind a particular door or door pairs, the handling apparatus typically contacts the door or door pairs with a gripper mechanism or other tool, and pushes it open as the gripper mechanism moves forward. The force of the handling apparatus overcomes the force of the spring or springs trying to keep the door or door pairs closed. Once the door is at least partially open (e.g., about 70° or about 65°, about 60°, or about 55° from vertical), a door hold-open mechanism(s) further opens the door or door pairs and holds the door or door pairs open so that the gripper mechanism of the handling apparatus no longer contacts the door or door pairs. In some embodiments, for example, the door hold-open mechanism(s) open the door or door pairs about an additional about 20° to about 35°. The handling apparatus then grips the container or other object, and removes it from the incubation device. Once the gripper mechanism and the object are clear of the door or door pairs, the door hold-open mechanism(s) is/are released and the spring(s) force(s) the door or door pairs closed. The internal environment of the incubation device is exposed to the external environment for only a short time, which reduces air transfer between the two environments. The size of a door or door pairs is typically just large enough to provide access for a single sample container or other object and the gripper mechanism.

Placing containers or other objects into incubation devices is very similar to the above-described process. However, instead of using gripper mechanisms to initially contact the door, the object disposed in the gripper mechanism contacts the door. Again, once the object pushes the door or door pairs open (e.g., about 70°, about 65°, about 60°, or about 55° from vertical), the door hold-open mechanism further opens the door or door pairs and holds the door or door pairs open so that the object no longer contacts the door or door pairs. When the door or door pairs is almost fully open (e.g., greater than about 75°, greater than about 85°, greater than about 90°, greater than about 95° or more), neither the robotic gripper mechanism nor the object is typically in contact with the door or door pairs. The robot then places the object on, e.g., a shelf, unclamps from the object, and leaves the object in the incubation device. Once the gripper mechanism is clear of the door or door pairs, the door hold-open mechanism(s) is/are released and the spring(s) force(s) the door or door pairs closed.

Thus, moving parts within the incubation devices are typically limited to carrousels and hold-open mechanisms that hold doors open. Reliability and serviceability are dramatically improved relative to pre-existing devices, because all the mechanical parts for opening the doors are part of the handling device and external to the incubation device. Should mechanical components fail, repairs are readily made without disturbing the internal environment within the incubation devices.

Individual shelves can include a number of embodiments to aid in accessing an object from a shelf and/or in placing and aligning the object on a particular shelf. For example, a section of the shelf, e.g., a second section, is smaller than a first section and typically smaller than an object, which provides an area on the object where the handling apparatus is able to freely grip the object without contacting the shelf. Sides of the shelves can also be angled, tapered or rounded to align the object on a particular shelf.

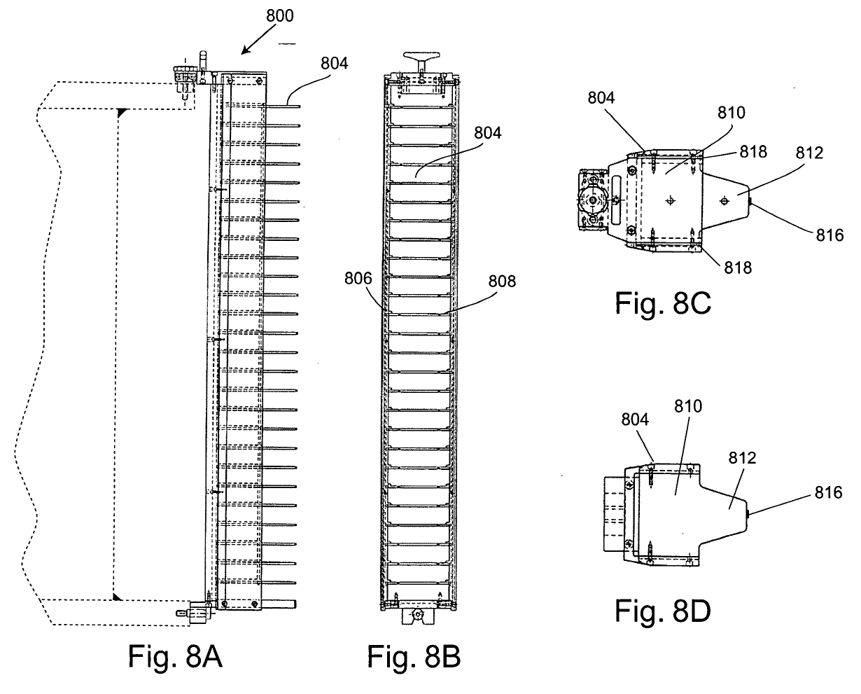

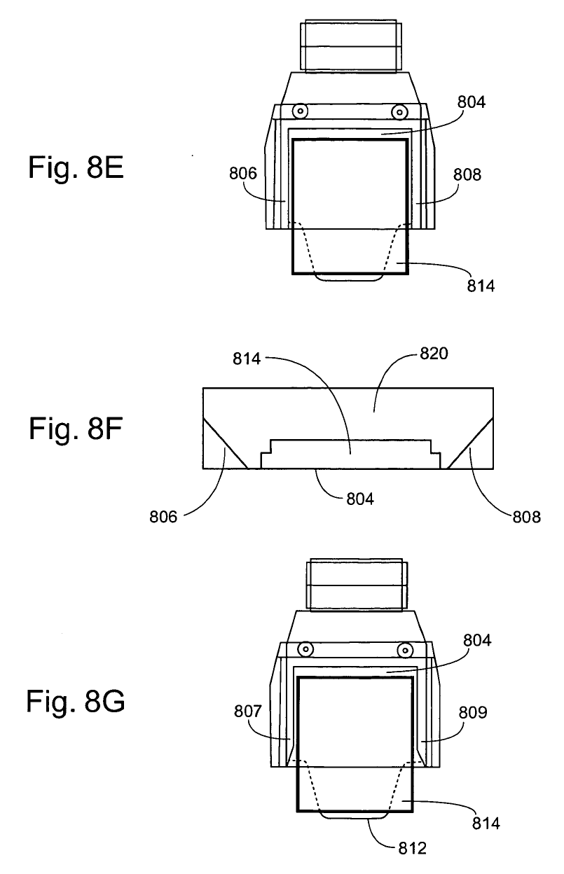

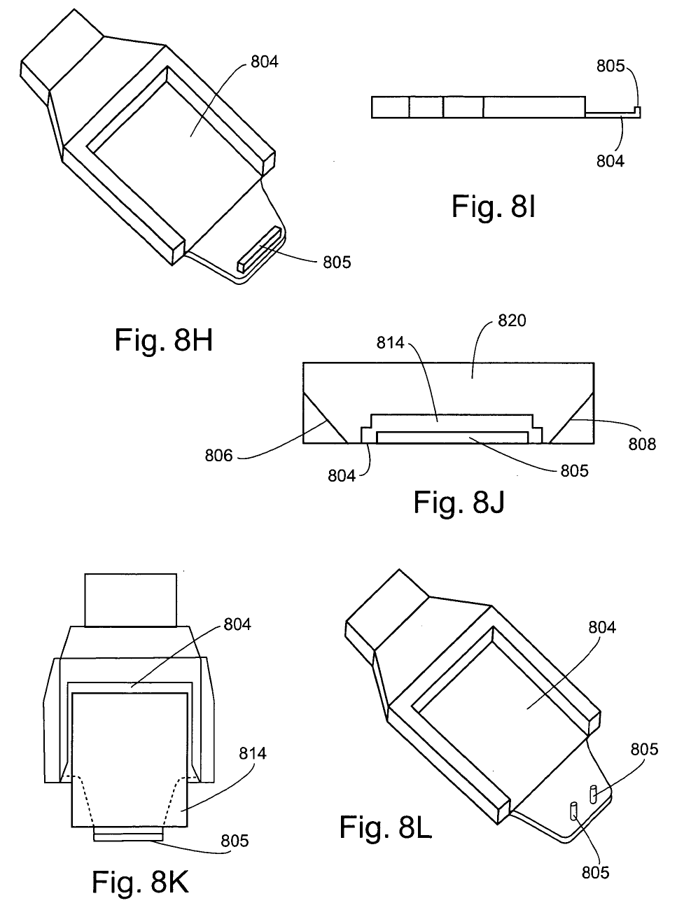

FIG. 8A schematically depicts a cutaway side view of vertical column of shelves 800 according to one embodiment of the invention. FIG. 8B schematically depicts a front cutaway view of vertical column of shelves 800 with sides of shelves 804 including first angled surface 806 and second angled surface 808. FIG. 8C schematically depicts a top cutaway view of shelf 804, which includes first section 810 and second section 812. FIG. 8D schematically depicts a bottom cutaway view shelf 804, including first section 810 and second section 812. Shelf 804 also includes identification label 816 in this embodiment. In addition, shelf 804 also includes sensor 818 in this embodiment. Identification label 816 and sensor 818 can also be located at various other locations on shelf 804.

FIG. 8E schematically depicts shelf 804 with first angled surface 806 and second angled surface 808 holding sample container 814. As shown, shelf 804 includes first angled surface 806 and second angled surface 808, which aid in aligning sample container 814 on the shelf 804. In one aspect, as shown in FIG. 8F first angled surface 806 and second angled surface 808 are tapered toward interior 820 of shelf 804. FIG. 8G schematically depicts another embodiment of the invention, where first angled surface 807 and second angled surface 809 are tapered toward second section 812. When a sample container or other object is placed on the shelf by, for example, a robotic gripper apparatus, the angled surfaces position the sample container in a precise desired position. Although shown in FIG. 8F with a gap between the bottom inward-facing portion of the angled surfaces and the sample container, in some embodiments the angled surfaces contact the sample container with little or no excess clearance, thereby providing for further precise positioning. The angled surfaces are shown as angled, but can also have other shapes (e.g., tapered, rounded) that will provide for precise positioning of a sample container or other object on the shelf.

In some embodiments, incubation device shelves include retaining features that are structured to retain objects in place on the shelves, e.g., when device carrousels are rotated, when objects positioned on shelves are agitated, or the like. Examples of retaining features are schematically depicted in FIGS. 8H-L. More specifically, FIGS. 8H and I schematically show shelf 804 from perspective and side elevational views, respectively. As shown, retaining feature 805 (shown as a retaining wall) extends from a surface of shelf 804. FIGS. 8J and K further show sample container 814 disposed on shelf 804 from front and top cutaway views, respectively. FIG. 8L schematically depicts retaining features 805 (shown as a retaining pegs) extending from a surface of shelf 804 from a perspective view according to another retaining feature embodiment.

The incubation devices of the invention can be used to incubate a wide variety of objects, including various types of sample containers. Some exemplary sample containers include microwell and deep-well plates (e.g., 6-well, 12-well, 24-well, 48-well, 96-well, 384-well, and 1536-well plates), other lab-ware, and the like. Other exemplary sample containers include, e.g., reaction blocks, reaction block carriers, petri dishes, test tubes, test tube racks, vials, crucibles, reaction vessels or flasks, hazardous material containers, medical devices or components, trays, etc. Certain reaction blocks and reaction block carriers are also described in, e.g., U.S. Pat. No. 6,682,703, entitled “PARALLEL REACTION DEVICES,” issued Jan. 27, 2004 to Micklash et al., and U.S. Ser. No. 60/351,821, entitled “DEVICES, SYSTEMS, AND METHODS OF MANIFOLDING MATERIALS,” filed Jan. 25, 2002 by Micklash et al., the disclosures of which are incorporated by reference in their entirety for all purpose. Microwell plates that are placed in the incubator can be covered by, for example, specimen plate lids such as those that are described in, e.g., U.S. Pat. No. 6,534,014, entitled “SPECIMEN PLATE LID AND METHOD OF USING,” issued Mar. 18, 2003 to Mainquist et al., which is incorporated by reference in its entirety for all purposes.

In certain embodiments, sensors are a feature of the incubation devices described herein. In one embodiment, a shelf includes a sensor for alignment with a door or door pairs, for interaction with a handling apparatus, etc. In another embodiment, sensors located on a door or door pairs, and/or the handling apparatus, and/or shelf can be used to signal an incubation device to open a particular door or close a particular door. Examples of sensors include optical sensors, photoelectric sensors, infrared sensors, position sensors, laser distance sensors, magnetic sensors and the like.

The incubation devices, or components thereof, of the invention are typically operably connected to one or more logic devices, such as computers or other information appliances. A logic device generally includes system software that directs, e.g., the gripper of the robotic armature to grasp selected sample containers, the movement of the robotic armature mass relative to the incubation device, or the like. For example, device components are optionally coupled to an appropriately programmed processor or computer which functions to instruct the operation of these instruments in accordance with preprogrammed or user input instructions, receive data and information from these instruments, and interpret, manipulate and report this information to the user.

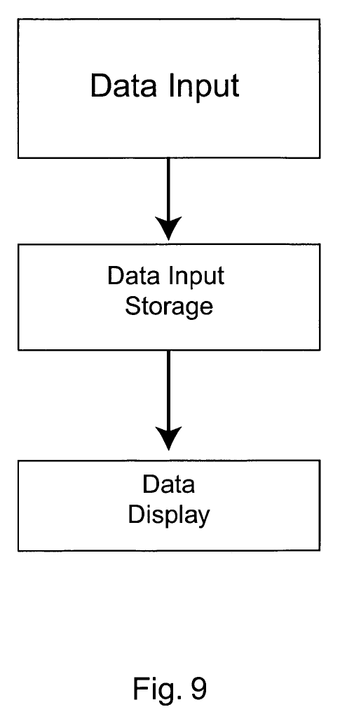

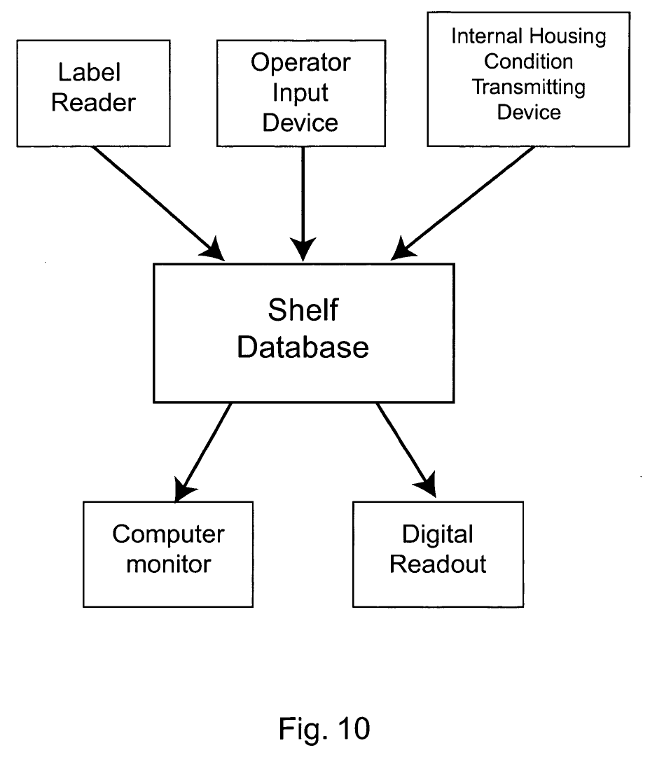

Computer systems in the incubation devices or systems can play many roles. The computer systems can acquire data, store data, and display data relevant to incubation devices of the invention (FIG. 9). One embodiment is diagramed in FIG. 10. The computer systems can provide instructions to operators, direction the operators or even exercise physical control over operator actions.

The computer is optionally, e.g., a PC (Intel x86 or Pentium chip-compatible DOS™, OS2™, WINDOWS™, WINDOWS NT™, WINDOWS95™, WINDOWS98™, WINDOWS2000™, WINDOWS XP™, a LINUX based machine, a MACINTOSH™, Power PC, or a UNIX based (e.g., SUN™ work station) machine) or other common commercially available computer, which is known to one of skill in the art. Software for performing the operations described herein is optionally easily constructed by one of skill using a standard programming language such as Visual basic, Fortran, Basic, Java, or the like. Any controller or computer optionally includes a monitor, which is often a cathode ray tube (“CRT”) display, a flat panel display (e.g., active matrix liquid crystal display, liquid crystal display), or others. Computer circuitry is often placed in a box that includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user.

The computer typically includes appropriate software for receiving user instructions, either in the form of user input into a set parameter fields, e.g., in a GUI, or in the form of preprogrammed instructions, e.g., preprogrammed for a variety of different specific operations. The software then converts these instructions to appropriate language for instructing, e.g., the control of internal housing conditions, the sample handling operations, movement of a particular shelf to a particular door of the incubation device, etc.

Data acquisition by the computer systems of the invention can include maintenance of an accurate sample inventory, movement of sample containers via a sampling handling device, internal housing conditions and the like. For example, when a change is made to a sample container in the incubation device, e.g., insertion of a sample container, movement of a sample container to a new location, removal of a sample container, and the like, the change can be documented in the inventory.

Inventory changes can be updated in the shelf database of the invention through operator input devices such as manual data entry using a computer keyboard. Shelf database information suitable for operator data entry includes, e.g., library names, sub-group descriptions, mother/daughter sample container designations, sample container types, compound structures or cell types, volumes removed per sample, volumes remaining per sample, and the like. Such data can be entered as a large batch of data in spreadsheet form. Alternately, such data can be entered in near real time on the operator's initiative or with computer prompting.

Inventory changes can also be updated to the shelf database to include sample container information by scanning, e.g., of bar code, labels. For example, identification labels, e.g., bar codes and the like, can be placed on the shelves and/or sample containers, which can make identification of sample containers, location of a particular shelf and data acquisition easier and more reliable. Sample container information suitable for scanned data input includes, e.g., sample container creation dates, sample container locations, sample container movement dates, sample container activity dates, and the like. Scanned data is often acquired real time and with high reliability.

Internal housing condition information can be captured by data input sources and transmitted to the computer for storage or output. Such data includes, e.g., temperature, humidity, gas composition, and the like. Instruments acting as data input sources can be simple, e.g., a thermosistor providing direct analog input of an internal housing temperature. More complex data input sources can be computerized instruments, e.g., analytical systems, in digital communication with the computer. Data acquisition can be continuous or intermittent depending on scientific and regulatory requirements. Thresholds, e.g., maximum or minimum temperatures, maximum and minimum humidity levels maximum, minimum gas composition levels and the like, can be established provide an alarm warning an operator of an unsuitable or hazardous condition.

Data acquired by the computer can be stored in databases, e.g., as a record of the past internal housing conditions or to establish the current status of shelves and/or sample containers. In one embodiment of the invention, a shelf database is compiled to reflect the current status of shelves and/or sample containers in the incubation device. The shelf database can include, e.g., status of a shelf (e.g., occupied or not occupied), library names, sub-group descriptions, mother/daughter sample container designations, sample container types, sample container creation dates, sample container locations, compound structures or cell types for each well, volumes for each well, and the like. Stored data can be transmitted to output devices for viewing or analysis.

The computers of the incubation device provide data output useful to, e.g., inform an operator of system conditions, prompt an operator to take actions, supply system documentation, and prevent errors. Data output devices of the invention include, e.g., liquid crystal (LC) displays, computer monitors, printers, and command interface boards connected to, e.g., lights, locks, and alarms.

Data output devices can inform an operator of system conditions. For example, a computer monitor or LC display can display the internal housing conditions, e.g., temperature, humidity, gas composition, and the like. An operator can then respond if degrading conditions indicate a system maintenance problem. The operator can decide to delay additional sample container movement operations until the desired internal housing conditions are met.

Data output consisting of procedural instructions for an operator is also a feature of the invention. The retrieval and storage methods, described herein, provide reliable sample handling and accurate inventories with careful attention to detail by the operator. Computer output of instructions and directions can help to insure proper functioning of the system.

The computer systems can transmit commands to take actions ensuring smooth operation of incubation device or systems of the invention. The computer system can be operably coupled, through an interface, to physical actuators, e.g., lights and alarms, to provide certain notice of system requirements to the operator. The computer can actuate alarms to warn of, e.g., open and/or malfunctioning doors, undesirable internal housing conditions, incorrect sample container reloading, and the like.

To further illustrate, the systems of the invention optionally include other components, e.g., in addition to handling apparatus, computer systems, and the like. In certain embodiments, for example, materials dispensing and/or removal systems are included in the systems of the invention. Additional details relating to some of these types of dispensing and/or removal systems and related methods, which are optionally adapted for use with the systems of the present invention are provided in, e.g., U.S. Provisional Patent Application No. 60/598,994, entitled “MULTI-WELL CONTAINER PROCESSING SYSTEMS, SYSTEM COMPONENTS, AND RELATED METHODS,” filed Aug. 4, 2004 by Micklash II et al. and International Publication No. WO 2004/091746, entitled “MATERIAL REMOVAL AND DISPENSING DEVICES, SYSTEMS, AND METHODS,” filed Apr. 7, 2004 by Micklash II et al., which are both incorporated by reference. In some embodiments, object positioning devices, such as multi-well container positioning devices are included in the systems of the invention. Certain of these positioning devices are described in, e.g., International Application No. PCT/US04/025079, entitled “MULTI-WELL CONTAINER POSITIONING DEVICES AND RELATED SYSTEMS AND METHODS,” filed Aug. 3, 2004 by Evans, International Publication No. WO 01/96880, entitled “AUTOMATED PRECISION OBJECT HOLDER,” filed Jun. 15, 2001 by Mainquist et al., and International Application No. PCT/US04/25170, entitled “NON-PRESSURE BASED FLUID TRANSFER IN ASSAY DETECTION SYSTEMS AND RELATED METHODS,” filed Aug. 3, 2004 by Evans et al., which are each incorporated by reference. Detection components are also optionally included in the systems of the invention and are described further in, e.g., Skoog et al., Principles of Instrumental Analysis, 5^{th} Ed., Harcourt Brace College Publishers (1998) and Currell, Analytical Instrumentation: Performance Characteristics and Quality, John Wiley & Sons, Inc. (2000), which are both incorporated by reference.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1. An incubation device, comprising:
(a) a housing that comprises at least one opening;
(b) at least one door operably connected to the housing and disposed in or proximal to the opening, wherein the door occludes the opening opening when the door is closed;
(c) a rotatable carousel that comprises at least one shelf; and,
(d) a rotational mechanism operably connected to the rotatable carousel, which rotational mechanism is configured to oscillate the rotatable carousel as the rotatable carousel is rotated to thereby agitate an object when the object is disposed on the shelf.

2. The incubation device of claim 1, wherein the housing comprises at least one additional door that allows access to an interior of the housing.

3. The incubation device of claim 1, wherein (b) comprises multiple doors arranged in at least one column and/or at least one row.

4. The incubation device of claim 1, wherein the door lacks an individual actuator mechanism that opens the door.

5. The incubation device of claim 1, wherein the shelf comprises a retaining feature that is structured to retain an object when the object is placed on the shelf.

6. The incubation device of claim 1, wherein the shelf is capable of moving into alignment with at least a portion of the opening.

7. The incubation device of claim 1, wherein the shelf comprises at least one identification label.

8. The incubation device of claim 1, wherein the shelf comprises at least one sensor.

9. The incubation device of claim 1, comprising:
(e) a door hold-open mechanism that comprises a member having at least one stop positioned proximal to the door, which stop is structured to move into contact with the door when the door is at least partially open to thereby hold the door open.

10. The incubation device of claim 9, wherein the stop comprises a prong attached to the member.

11. The incubation device of claim 10, comprising an actuating device that is configured to move the door hold-open mechanism.

12. The incubation device of claim 1, wherein (b) comprises at least one door pair, and wherein the incubation device comprises (e) at least first and second door hold-open mechanisms,
wherein the first door hold-open mechanism comprises a first member having at least a first stop positioned proximal to the first door, which first stop contacts the first door when the first member moves in a upward direction and the first door is at least partially open to thereby hold the first door open, and
wherein the second door hold-open mechanism comprises a second member having at least a second stop positioned proximal to the second door, which second stop contacts the second door when the second member moves in a downward direction and the second door is at least partially open to thereby hold the second door open.

13. The incubation device of claim 12, wherein opposing sides of the first and second doors are hinged such that movement of the first member of the first door hold-open mechanism in a first direction causes the first stop to hold the first door open when the first door is at least partially open, and movement of the second member of the second door hold-open mechanism in a second direction, that is opposite from the first direction, causes the second stop to hold the second door open when the second door is at least partially open.

14. The incubation device of claim 12, wherein either or both of the first stop and the second stop comprises a prong.

15. The incubation device of claim 12, comprising at least one actuating device that is configured to move at least one of the door hold-open mechanisms.

16. The incubation device of claim 12, comprising a plurality of door pairs arranged in a vertical column, each of which door pairs comprises a first door and a second door and occludes an opening or a portion of an opening in the housing when the both the first door and the second door pair of the door pair are closed.

17. The incubation device of claim 16, wherein the first door hold-open mechanism comprises multiple stops that are each positioned adjacent to a different first door, wherein movement of the first member of the door hold-open mechanism results in a stop contacting any first door that is at least partially open, thereby holding open any first door that is at least partially open.

18. The incubation device of claim 16, wherein the second door hold-open mechanism comprises multiple stops that are each positioned adjacent to a different second door, wherein movement of the second member in a direction opposite to the direction of movement of the first member results in a stop contacting any second door that is at least partially open, thereby holding open any second door that is at least partially open.

19. The incubation device of claim 1, comprising at least one restraining mechanism operably connected to the door, which restraining mechanism is configured to close the door in the absence of an opposing force applied on the door.

20. The incubation device of claim 19, wherein the restraining mechanism comprises one or more springs.

21. The incubation device of claim 1, comprising at least one controller operably connected to the housing, which controller is configured to control one or more internal housing conditions.

22. The incubation device of claim 21, wherein the internal housing conditions comprise one or more of: temperature, humidity, or gas composition.

23. The incubation device of claim 1, comprising at least one access panel operably connected to the housing, which access panel comprises the door.

24. The incubation device of claim 23, wherein the access panel is operably connected to the housing via at least one hinge.

25. The incubation device of claim 23, wherein the access panel comprises at least one gasket.

26. The incubation device of claim 23, wherein the access panel comprises a lock and/or a latch.

27. The incubation device of claim 23, wherein the access panel is disposed in an additional door in the housing.

28. A system that comprises the incubation device of claim 1.

29. The system of claim 28, comprising at least one handling apparatus disposed substantially external to the housing, which handling apparatus is configured to move objects into and out of the incubation device through the door pairs.

30. The system of claim 29, comprising at least one logic device operably connected at least to the handling apparatus, which logic device comprises one or more logic instructions that direct movement of the handling apparatus.

31. The system of claim 29, wherein the handling apparatus comprises at least one robotic armature.

32. The system of claim 29, wherein the handling apparatus comprises at least one gripper mechanism that is structured to grip one or more objects.

33. The system of claim 28, comprising at least one computer system operably connected to the incubation device, which computer system comprises one or more of: a data input source, a data storage location, or a data output device.

34. The system of claim 33, wherein the data input source comprises one or more of: a label reader, an operator input device, or an internal housing condition transmitting device.

35. The system of claim 33, wherein the data storage location comprises a shelf database.

36. The system of claim 33, wherein the data output device comprises one or more of: a computer monitor or a digital readout.

37. An incubation device, comprising:
(a) a housing that comprises at least one opening;
(b) at least one door pair comprising a first door and a second door, wherein a top portion of the first door and a bottom portion of the second door are pivotably attached to the housing and the opening is occluded when both the first door and the second door are closed; and,
(c) at least a first and a second door hold-open mechanism,
wherein the first door hold-open mechanism comprises a first member having at least a first stop positioned proximal to the first door, which first stop contacts the first door when the first member moves in a upward direction and the first door is at least partially open to thereby hold the first door open, and
wherein the second door hold-open mechanism comprises a second member having at least a second stop positioned proximal to the second door, which second stop contacts the second door when the second member moves in a downward direction and the second door is at least partially open to thereby hold the second door open.

38. The incubation device of claim 37, wherein the housing further comprises at least one access door that allows access to an interior of the housing.

39. The incubation device of claim 37, wherein the first and second doors open independently of one another.

40. The incubation device of claim 37, wherein the doors comprise a non-linear interface with one another.

41. The incubation device of claim 37, wherein the doors are structured to accommodate at least one deep-well plate.

42. The incubation device of claim 37, wherein the doors lack individual actuator mechanisms that open the doors.

43. The incubation device of claim 37, wherein the first door and the second door are each rotatably attached to the housing with a hinge.

44. The incubation device of claim 37, wherein the first stop comprises a prong attached to the first member and the second stop comprises a prong attached to the second member.

45. The incubation device of claim 37, comprising at least one actuating device that is configured to move at least one of the door hold-open mechanisms.

46. The incubation device of claim 37, comprising a plurality of door pairs arranged in a vertical column, each of which door pairs comprises a first door and a second door and occludes an opening or a portion of an opening in the housing when the both the first door and the second door pair of the door pair are closed.

47. The incubation device of claim 46, wherein the plurality of door pairs is arranged in a plurality of columns and/or at least one row.

48. The incubation device of claim 46, wherein the first door hold-open mechanism comprises multiple stops that are each positioned adjacent to a different first door, wherein upward movement of the first member of the door hold-open mechanism results in a stop contacting any first door that is at least partially open, thereby holding open any first door that is at least partially open.

49. The incubation device of claim 46, wherein the second door hold-open mechanism comprises multiple stops that are each positioned adjacent to a different second door, wherein downward movement of the second member results in a stop contacting any second door that is at least partially open, thereby holding open any second door that is at least partially open.

50. The incubation device of claim 37, comprising one or more restraining mechanisms operably connected to one or more of the doors, which restraining mechanisms are configured to close the doors in the absence of opposing forces applied to the doors.

51. The incubation device of claim 50, wherein the restraining mechanisms comprise one or more springs.

52. The incubation device of claim 37, comprising at least one shelf disposed within the housing.

53. The incubation device of claim 52, wherein the shelf is configured to agitate an object when the object is placed on the shelf.

54. The incubation device of claim 52, wherein the shelf comprises a retaining feature that is structured to retain an object when the object is placed on the shelf.

55. The incubation device of claim 52, wherein the shelf is capable of moving into alignment with at least a portion of the opening.

56. The incubation device of claim 52, wherein the shelf comprises at least one angled surface that is structured to align an object on the shelf, when the object is placed on the shelf.

57. The incubation device of claim 52, wherein the shelf comprises at least one identification label.

58. The incubation device of claim 52, wherein the shelf comprises at least one sensor.

59. The incubation device of claim 52, comprising multiple shelves arranged in a rotatable carrousel.

60. The incubation device of claim 59, comprising at least one rotational mechanism operably connected to the rotatable carrousel, which rotational mechanism is configured to rotate the rotatable carrousel in one or more selectable modes.

61. The incubation device of claim 60, wherein at least one of the selectable modes comprises an oscillation of the rotatable carrousel as the rotatable carrousel is rotated to thereby agitate an object when the object is disposed on the shelf.

62. The incubation device of claim 37, comprising at least one controller operably connected to the housing, which controller is configured to control one or more internal housing conditions.

63. The incubation device of claim 62, wherein the internal housing conditions comprise one or more of: temperature, humidity, or gas composition.

64. The incubation device of claim 37, comprising at least one access panel operably connected to the housing, which access panel comprises the door pairs.

65. The incubation device of claim 64, wherein the access panel is operably connected to the housing via at least one hinge.

66. The incubation device of claim 64, wherein the access panel comprises at least one gasket.

67. The incubation device of claim 64, wherein the access panel comprises a lock and/or a latch.

68. The incubation device of claim 64, wherein the access panel is disposed in an additional door in the housing.

69. A system that comprises the incubation device of claim 37.

70. The system of claim 69, comprising at least one handling apparatus disposed substantially external to the housing, which handling apparatus is configured to move objects into and out of the incubation device through the door pairs.

71. The system of claim 70, comprising at least one logic device operably connected at least to the handling apparatus, which logic device comprises one or more logic instructions that direct movement of the handling apparatus.

72. The system of claim 70, wherein the handling apparatus comprises at least one robotic armature.

73. The system of claim 70, wherein the handling apparatus comprises at least one gripper mechanism that is structured to grip at least one object.

74. The system of claim 69, comprising at least one computer system operably connected to the incubation device, which computer system comprises one or more of: a data input source, a data storage location, or a data output device.

75. The system of claim 74, wherein the data input source comprises one or more of: a label reader, an operator input device, or an internal housing condition transmitting device.

76. The system of claim 74, wherein the data storage location comprises a shelf database.

77. The system of claim 74, wherein the data output device comprises one or more of: a computer monitor or a digital readout.

78. A method of accessing an incubation device, the method comprising:
(a) contacting a door pairs of the incubation device such that door hold-open mechanisms of the incubation device hold the doors open; and, (b) placing an object on, or removing the object from, a shelf disposed within the incubation device, thereby accessing the incubation device.

79. The method of claim 78, comprising performing (a) and (b) using a robotic armature of a handling apparatus.

80. A method of agitating an object, the method comprising:
(a) providing an incubation device that houses a rotatable carousel that comprises at least one shelf having the object positioned thereon;
(b) rotating the rotatable carousel; and,
(c) oscillating the rotatable carousel as the rotatable carousel is rotated, thereby agitating the object.
